(19) United States
(12) Patent Application Publication
(10) Pub. No.: US 2002/0176836 A9
(43) Pub. Date: Nov. 28, 2002

(54) Thickened hair composition comprising a fixing polymer and pulverulent compound

(76) Inventors: Emmanuelle Belli, Asnieres (FR); Herve Jourdan, Saint-Cyr (FR); Dorothee Pasquet, Bois Colombes (FR)

Correspondence Address: FINNEGAN, HENDERSON, FARABOW, GARRETT &, DUNNER LLP, 1300 I STREET, NW, WASHINGTON, DC, 20005, US

(21) Appl. No.: 09/799,911

(22) Filed: Mar. 7, 2001

(30) Foreign Application Priority Data
Mar. 7, 2000 (FR) 00 02907

(51) Int. Cl. A61K 7/06; A61K 7/11

(52) U.S. Cl. 424/70.16; 424/70.13

(57) ABSTRACT

A cosmetic hair composition comprising, in a cosmetically acceptable medium, at least one thickener, at least one fixing polymer, and at least one pulverulent material. The at least one thickener comprises at least one compound chosen from: (i) natural polymers, and modified natural polymers chosen from xanthan gums, scleroglucan gums, gellan gums, rhamsan gums, alginates, maltodextrins, starches and their derivatives, karaya gums, carob flours, modified and unmodified guar gums and derivatives thereof, and celluloses and derivatives thereof; (ii) crosslinked copolymers of at least one acid chosen from acrylic acid and methacrylic acid, (iii) crosslinked thickening polyacrylamides and (iv) associative polymers comprising at least one hydrophilic unit and at least one fatty chain. The at least one fixing polymer is a non-crosslinked polymer. The at least one pulverulent material is formed from at least one non-lamellar insoluble material.

The invention relates to a cosmetic hair composition, such as, for example, a gel, comprising at least one thickening polymer, at least one fixing polymer and at least one insoluble pulverulent material. The term “insoluble” means that the compound is soluble in the composition in an amount less than 0.1% by weight, the solubility representing the amount of compound capable of dissolving in 100 grams of the composition.

The invention also relates to a cosmetic haircare process comprising the application of this composition to the hair, as well as to processes that result in at least one benefit chosen from fixing and holding a hairstyle.

For the purposes of the present invention, the term “gel” means a composition which is initially liquid, and which is thickened by means of at least one agent with at least one property chosen from thickening and gelling properties, finally having a minimum viscosity of approximately 160 cps, such as 250 cps, at 25° C. (Rheomat 180 viscometer—spindle No. 2—reading after 30 seconds).

Products for fixing and holding a hairstyle can be in various forms, such as in the form of gels, are known. Although they often allow long-lasting fixing of the hairstyle, they nevertheless can have various drawbacks.

For example, many consumers complain that the gel remains stuck to the hands during application, that it takes too long to dry on the hair, which may lead to a collapse of the hairstyle if the gel is applied at the end to dry hair, and that it gives the hair an artificial appearance, such as, for example, an unnatural sheen.

To solve the above problems, it has already been proposed to produce cosmetic hair products comprising a fixing polymer, a thickener and a pulverulent agent. However, the compositions known to date comprising these constituents may still not be entirely satisfactory, since the hairstyle may still lack a natural look and since the gels may still stick to the hands too much during application and may dry too slowly.

The problem posed by the invention is that of providing a cosmetic hair composition, such as, for example, in the form of a gel, which is better than the compositions of the prior art, and for example, may have at least one of the following properties: leaves the hands clean after it has been applied to the hair, makes possible an application to dry hair without destructuring the hairstyle, dries quickly, holds the shape of the hairstyle strongly and may be distributed homogeneously.

The inventors have discovered, surprisingly and unexpectedly, that by carefully selecting the fixing polymer, the thickener and the pulverulent agent to make the cosmetic hair composition, it is possible to solve at least one of the problems posed above.

A subject of the invention is a cosmetic hair composition comprising (a) at least one thickener, (b) at least one fixing polymer and (c) at least one pulverulent material, in a cosmetically acceptable medium, wherein,

the at least one thickener comprises at least one compound chosen from (i) natural polymers and modified natural polymers, (ii) crosslinked copolymers of at least one acid chosen from acrylic acid and methacrylic acid, (iii) crosslinked thickening polyacrylamides and (iv) associative polymers comprising at least one hydrophilic unit and at least one fatty chain;

the at least one fixing polymer is a non-crosslinked polymer, and

the at least one pulverulent material is formed from at least one non-lamellar insoluble material.

Another subject of the invention relates to a cosmetic haircare process, such as, for example, a process for fixing and holding a hairstyle using the said composition.

Another subject of the invention relates to the process of applying the composition in an amount effective for holding and shaping a hairstyle.

As used in the specification and claims, the general expression “copolymers of monomer A and monomer B” means copolymers derived from monomer A and monomer B. Thus, for example, the expression “copolymers of alkyl acrylate and of urethane” means copolymers derived from alkyl acrylate and urethane.

According to the invention, the expression “modified natural thickener” means any thickening polymer obtained by simple chemical modification of the natural polymer itself.

Polyquaternium-10 is a polymeric quaternary ammonium salt of hydroxyethylcellulose reacted with a trimethyl ammonium substituted epoxide, as defined in the International Cosmetic Ingredient Dictionary and Handbook, Seventh Edition, Volume 2 (1997) pages 1087-1088, these pages being specifically incorporated by reference herein.

One aspect of the invention relates to a cosmetic hair composition, comprising:

in a cosmetically acceptable medium

(a) at least one thickener,

(b) at least one fixing polymer, and

(c) at least one pulverulent material, wherein,

the at least one thickener is chosen from,

(i) natural polymers and modified natural polymers chosen from xanthan gums, scleroglucan gums, gellan gums, rhamsan gums, alginates, maltodextrins, starches and their derivatives, karaya gums, carob flours, modified and unmodified guar gums and derivatives thereof, and celluloses and derivatives thereof,

(ii) crosslinked copolymers of at least one acid chosen from acrylic acid and methacrylic acid,

(iii) crosslinked thickening polyacrylamides and

(iv) associative polymers comprising at least one hydrophilic unit and at least one fatty chain;

the at least one fixing polymer is a non-crosslinked polymer,

the at least one pulverulent material is formed from at least one non-lamellar insoluble material, and

with the proviso that said at least one thickener is not Polyquaternium-10.

Another aspect of the invention relates to a gel comprising,

in a cosmetically acceptable medium

(a) at least one thickener,

(b) at least one fixing polymer, and

(c) at least one pulverulent material, wherein,

the at least one thickener is chosen from,

(i) natural polymers and modified natural polymers chosen from xanthan gums, scleroglucan gums, gellan gums, rhamsan gums, alginates, maltodextrins, starches and their derivatives, karaya gums, carob flours, modified and unmodified guar gums and derivatives thereof, and celluloses and derivatives thereof,

(ii) crosslinked copolymers of at least one acid chosen from acrylic acid and methacrylic acid,

(iii) crosslinked thickening polyacrylamides and

(iv) associative polymers comprising at least one hydrophilic unit and at least one fatty chain;

the at least one fixing polymer is a non-crosslinked polymer, and

the at least one pulverulent material is formed from at least one non-lamellar insoluble material.

A further aspect of the invention relates to a process comprising applying a composition to hair, wherein said composition comprises, in a cosmetically acceptable medium

(a) at least one thickener,

(b) at least one fixing polymer, and

(c) at least one pulverulent material, wherein,

the at least one thickener is chosen from,

(i) natural polymers and modified natural polymers chosen from xanthan gums, scleroglucan gums, gellan gums, rhamsan gums, alginates, maltodextrins, starches and their derivatives, karaya gums, carob flours, modified and unmodified guar gums and derivatives thereof, and celluloses and derivatives thereof,

(ii) crosslinked copolymers of at least one acid chosen from acrylic acid and methacrylic acid,

(iii) crosslinked thickening polyacrylamides and

(iv) associative polymers comprising at least one hydrophilic unit and at least one fatty chain;

the at least one fixing polymer is a non-crosslinked polymer,

the at least one pulverulent material is formed from at least one non-lamellar insoluble material, and

with the proviso that said at least one thickener is not Polyquaternium-10.

Yet another aspect of the invention relates to a process for at least one of fixing and holding a hair style comprising applying an effective amount of a composition to said hair, said composition comprising:

in a cosmetically acceptable medium

(a) at least one thickener,

(b) at least one fixing polymer, and

(c) at least one pulverulent material, wherein,

the at least one thickener is chosen from,

(i) natural polymers and modified natural polymers chosen from xanthan gums, scleroglucan gums, gellan gums, rhamsan gums, alginates, maltodextrins, starches and their derivatives, karaya gums, carob flours, modified and unmodified guar gums and derivatives thereof, and celluloses and derivatives thereof,

(ii) crosslinked copolymers of at least one acid chosen from acrylic acid and methacrylic acid,

(iii) crosslinked thickening polyacrylamides and

(iv) associative polymers comprising at least one hydrophilic unit and at least one fatty chain;

the at least one fixing polymer is a non-crosslinked polymer, and

the at least one pulverulent material is formed from at least one non-lamellar insoluble material.

Another aspect of the invention relates to a cosmetic hair composition, comprising:

in a cosmetically acceptable medium

(a) at least one thickener,

(b) at least one fixing polymer, and

(c) at least one pulverulent material, wherein,

the at least one thickener is chosen from,

(i) natural polymers and modified natural polymers of natural microbial origin

(ii) crosslinked copolymers of at least one acid chosen from acrylic acid and methacrylic acid,

(iii) crosslinked thickening polyacrylamides and

(iv) associative polymers comprising at least one hydrophilic unit and at least one fatty chain;

the at least one fixing polymer is a non-crosslinked polymer, and

the at least one pulverulent material is formed from at least one non-lamellar insoluble material.

Another aspect of the invention, moreover, relates to a cosmetic hair composition, comprising:

in a cosmetically acceptable medium

(a) at least one thickener,

(b) at least one fixing polymer, and

(c) at least one pulverulent material, wherein,

the at least one thickener is chosen from,

(i) natural polymers and modified natural polymers chosen from xanthan gums, scleroglucan gums, gellan gums, rhamsan gums, alginates, maltodextrins, starches and their derivatives, karaya gums, carob flours, modified and unmodified guar gums and derivatives thereof,

(ii) crosslinked copolymers of at least one acid chosen from acrylic acid and methacrylic acid,

(iii) crosslinked thickening polyacrylamides and

(iv) associative polymers comprising at least one hydrophilic unit and at least one fatty chain;

the at least one fixing polymer is a non-crosslinked polymer, and

the at least one pulverulent material is formed from at least one non-lamellar insoluble material.

Yet another aspect of the invention relates to a process comprising applying a composition to hair, wherein said composition comprises, in a cosmetically acceptable medium

(a) at least one thickener,

(b) at least one fixing polymer, and

(c) at least one pulverulent material, wherein,

the at least one thickener is chosen from,

(i) natural polymers and modified natural polymers chosen from xanthan gums, scleroglucan gums, gellan gums, rhamsan gums, alginates, maltodextrins, starches and their derivatives, karaya gums, carob flours, modified and unmodified guar gums and derivatives thereof,

(ii) crosslinked copolymers of at least one acid chosen from acrylic acid and methacrylic acid,

(iii) crosslinked thickening polyacrylamides and

(iv) associative polymers comprising at least one hydrophilic unit and at least one fatty chain;

the at least one fixing polymer is a non-crosslinked polymer, and

the at least one pulverulent material is formed from at least one non-lamellar insoluble material.

Natural and modified natural thickeners which are suitable for the invention include, for example, xanthan gums, scleroglucan gums, gellan gums, rhamsan gums, alginates, maltodextrins, starches and their derivatives, karaya gums, carob flours, modified and unmodified guar gums and derivatives thereof, such as hydroxypropylguar, and celluloses and derivatives thereof. Thickening celluloses and derivatives thereof (e.g., hydroxyethylcellulose, hydroxypropylcellulose and carboxymethylcellulose), guar gums and derivatives thereof (e.g., hydroxypropylguar) and gums of natural microbial origin (e.g., xanthan gums and scleroglucan gums) may be used.

The crosslinked copolymers of at least one acid chosen from acrylic acids and methacrylic acids are, for example, acrylic acid/ethyl acrylate copolymers and carboxyvinyl polymers. Such polymers are, for example, the “carbomer” products (CTFA) sold by the company Goodrich under the name Carbopol.

The crosslinked thickening polyacrylamides may be chosen, for example, from:

crosslinked 2-acrylamido-2-methylpropanesulphonic homopolymers,

optionally crosslinked copolymers of acrylamides and of ammonium acrylates,

crosslinked copolymers of methacryloyl-oxyethyltrimethylammonium chloride and of at least one group chosen from acrylamides and methacrylamides,

crosslinked, at least partially neutralized copolymers of acrylamides and of 2-acrylamido-2-methylpropanesulphonic acids.

As crosslinked copolymers of acrylamide/ammonium acrylate that are used in accordance with the present invention, mention may be made, for example, of acrylamide/ammonium acrylate copolymers (5/95 by weight) crosslinked with at least one crosslinking agent containing polyolefinic unsaturation, such as, for example, divinylbenzene, tetraallyloxyethane, methylenebisacrylamide, diallyl ether, polyallylpolyglyceryl ethers and allyl ethers of alcohols of the sugar series, such as, for example, erythritol, pentaerythritol, arabitol, mannitol, sorbitol and glucose.

Similar copolymers are described and prepared in French patent FR-2 416 723 and U.S. Pat. No. 2,798,053 and U.S. Pat. No. 2,923,692, the disclosures of all of which are specifically herein incorporated by reference.

These crosslinked copolymers of acrylamide/ammonium acrylate may be, for example, in the form of a water-in-oil emulsion comprising about 30% by weight of the crosslinked copolymers, about 25% by weight of liquid paraffin, about 4% by weight of a mixture of sorbitan stearate and at least one hydrophilic ethoxylated derivative, and 41% by weight of water. Such an emulsion is sold under the name “Bozepol C” by the company Hoechst.

The crosslinked copolymers of acrylamide and of 2-acrylamido-2-methylpropanesulphonic acid, that may be used in accordance with the present invention, may be copolymers crosslinked with compounds containing polyolefinic unsaturation, such as those mentioned above, and at least partially neutralized with at least one neutralizing agent such as, for example, sodium hydroxide, potassium hydroxide, aqueous ammonia and amines such as triethanolamine and monoethanolamine.

They can be prepared, for example, by copolymerizing acrylamide and sodium 2-acrylamido-2-methylpropanesulphonate via a radical-mediated route by means of initiators such as, for example, azobisisobutyronitrile and by precipitation in alcohol such as, for example, tert-butanol.

Copolymers may be obtained, for example, by copolymerization of an amount generally ranging from 70 mol % to 55 mol % of acrylamide and of an amount generally ranging from 30 mol % to 45 mol % of sodium 2-acrylamido-2-methylpropanesulphonate. The crosslinking agent is used at concentrations generally ranging from 10⁻⁴ to 4×10⁻⁴ mol per mole of the mixture of monomers.

These specific copolymers may be, for example, incorporated into the compositions of the invention in the form of water-in-oil emulsions containing an amount generally ranging from 35% to 40% by weight of the copolymers, an amount generally ranging from 15% to 25% by weight of a mixture of C₁₂-C₁₃ isoparaffinic hydrocarbons, an amount generally ranging from 3% to 8% by weight of polyethylene glycol lauryl ether containing about 7 mol of ethylene oxide, and water. Such an emulsion is sold under the name “Sepigel 305” by the company SEPPIC.

The crosslinked copolymers of methacryloyl-oxyethyltrimethylammonium chloride and of at least one group chosen from acrylamides and methacrylamides, according to the invention are, for example, copolymers obtained by copolymerization of acrylamide and of dimethylaminoethyl methacrylate quaternized with methyl chloride, followed by crosslinking with at least one compound containing olefinic unsaturation, such as, for example, methylenebisacrylamide.

Crosslinked acrylamide/methacryloyloxyethyltrimethylammonium chloride copolymers (about 50/50 by weight) in the form of a dispersion containing about 50% by weight of the said copolymers in mineral oil may be used. This dispersion is sold under the name “Salcare SC92” by the company Allied Colloids.

The non-crosslinked copolymers of methacrylamide and of methacryloyloxyethyltrimethylammonium chloride are, for example, the products sold under the trade names Rohagit KF 400 and KF720 by the company Rohm & Haas.

The associative polymers of the invention may belong to the polyurethane class of polymers.

Associative polyurethanes may be block copolymers comprising in the chain both hydrophilic sequences, which may be of a polyoxyethylenated nature, and hydrophobic sequences, which may be at least one sequence chosen from aliphatic chain units, cycloaliphatic chain units and aromatic chain units.

For example, these polymers may comprise at least two hydrocarbon-based lipophilic chains containing from 6 to 30 carbon atoms, separated by at least one hydrophilic sequence; the hydrocarbon-based chains can be pendent chains and chains at the end of the hydrophilic sequence. For example, it is possible for at least one pendent chain to be present. In addition, the polymer can comprise a hydrocarbon-based chain at at least one end of a hydrophilic sequence.

The polymers can be sequenced in triblock and multiblock form. Hydrophobic sequences can thus be at each end of the chain (for example, a triblock copolymer with a hydrophilic central sequence). The hydrophobic sequences may also be distributed both at the ends and in the chain (for example, a multiblock copolymer). The polymers can also be graft polymers and starburst polymers.

For example, the polymers may be triblock copolymers whose hydrophilic sequence is a polyoxyethylenated chain comprising generally from 50 to 1 000 oxyethylene groups. In general, the associative polyurethanes comprise a urethane linkage between the hydrophilic sequences, which explains the origin of the name.

Associative polyurethanes may also include polymers whose hydrophilic sequences are linked by other chemical linkages to the lipophilic sequences.

As examples of associative polymers which can be used in the invention, mention may be made of the polymer C₁₆-OE₁₂₀-C₁₆ sold by the company Hüls (under the name Serad FX1100, this molecule containing a urethane function and having a weight-average molecular weight of 1 300), OE being an oxyethylene unit. Associative polymers which can also be used are Rheolate 205 containing a urea function, sold by the company Rheox, Rheolate 208, and Reholate 204. These associative polyurethanes are sold in pure form.

The product DW 1206B from Rhom & Haas, which contains C₂₀ alkyl chains and urethane linkages, sold at a concentration of 20% solids in water, can also be used.

Solutions and dispersions of these polymers, for example, in mediums chosen from water and aqueous-alcoholic mediums may also be used. Examples of such polymers include Serad FX1010 and Serad FX1035 sold by the company Hüls, and Rheolate 255, Rheolate 278 and Rheolate 244 sold by the company Rheox. The products DW 1206F and DW 1206J can also be used, as can Acrysol RM 184 and Acrysol 44 (Aculyn 44) from the company Rhom & Haas.

The associative polymers which can be used in the invention are, for example, those described in the article by G. Fonnum, J. Bakke and Fk. Hansen—Colloid Polym. Sci 271, 380.389 (1993), the disclosure of which is specifically herein incorporated by reference.

The associative polyurethanes may be chosen, for example, from:

polyurethane polyethers (a) which can be obtained by polycondensation of at least three compounds comprising (i) at least one polyethylene glycol comprising from 50 to 500 mol of ethylene oxide, (ii) at least one C₈-C₃₀ fatty alcohol and (iii) at least one diisocyanate and,

polyurethane polyethers (b) which can be obtained by polycondensation of at least three compounds comprising (i) at least one polyethylene glycol comprising from 50 to 500 mol of ethylene oxide, (ii) at least one C₈-C₃₀ fatty alcohol other than that of the polyurethane polyether (a) and (iii) at least one diisocyanate.

For example, polyurethane polyethers (a) and (b) for which the polyethylene glycol comprises an amount generally ranging from 150 to 180 mols, such as 150 or 180 mol, of ethylene oxide may be used.

Further examples include polyurethane polyethers (a) and (b) for which the diisocyanate is methylenebis(4-cyclohexyl isocyanate).

Examples of compositions according to the invention include compositions as defined above, for which the polyurethane polyether (a) is obtained by polycondensation of at least three compounds comprising polyethylene glycol containing 150 or 180 mol of ethylene oxide, stearyl (C18) alcohol and methylenebis(4-cyclohexyl isocyanate), and the polyurethane polyether (b) is obtained by polycondensation of at least three compounds comprising polyethylene glycol containing 150 or 180 mol of ethylene oxide, decyl (C10) alcohol and methylenebis(4-cyclohexyl isocyanate).

Among the polyurethane polyethers (a), mention may be made of the product sold by the company Rohm & Haas under the trade name:

Aculyn 46, which is a polycondensate comprising at least, as elements, polyethylene glycol containing 150 or 180 mol of ethylene oxide, stearyl alcohol and methylenebis(4-cyclohexyl isocyanate) (SMDI), at 15% by weight in a matrix of maltodextrin (4%) and water (81%).

Among the polyurethane polyethers (b), mention may be made of the product sold by the company Rohm & Haas under the trade name:

Aculyn 44, which is a polycondensate comprising at least, as elements, polyethylene glycol containing 150 or 180 mol of ethylene oxide, decyl alcohol and methylenebis(4-cyclohexyl isocyanate) (SMDI), at 35% by weight in a mixture of propylene glycol (39%) and water (26%).

Viscophobe DB 1000 (Union Carbide) may also be used as an associative polyurethane.

Examples of other associative polymers which may be used include (meth)acrylic acid/C10-C30 alkyl acrylate copolymers such as Pemulen TR1 from Goodrich, nonionic celluloses containing a fatty chain, such as Natrosol Plus Grade 330 CS from Aqualon and cationic celluloses containing a fatty chain, such as Crodacel QS from Croda.

The non-crosslinked fixing polymer (b) is generally chosen from anionic, cationic, amphoteric and nonionic fixing polymers and mixtures thereof. A fixing polymer is a polymer which is capable of at least one of holding and fixing the shape of a hairstyle.

These fixing polymers may be used in dissolved form and alternatively in the form of a dispersion of solid polymer particles.

The cationic, anionic, amphoteric and nonionic fixing polymers (b) that may be used in accordance with the invention are described below.

The cationic fixing polymers which may be used according to the present invention are, for example, chosen from polymers containing primary, secondary, tertiary and quaternary amine groups forming part of the polymer chain and polymers containing primary, secondary, tertiary and quaternary amine groups directly attached to the chain, and having a molecular weight ranging from 500 to about 5 000 000, such as from 1 000 to 3 000 000.

Among these polymers, mention may be made of the following cationic polymers:

(1) homopolymers and copolymers derived from at least one group chosen from acrylic esters, methacrylic esters and amides and containing at least one of the units of the following formulae:

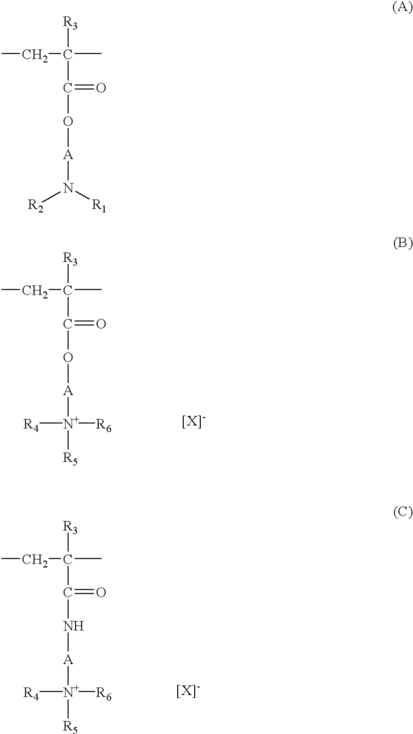

in which:

R₃ is chosen from hydrogen and CH₃ radicals;

A is chosen from linear and branched alkyl group of 1 to 6 carbon atoms and hydroxyalkyl groups of 1 to 4 carbon atoms;

R₄, R₅ and R₆, which may be identical or different, are chosen from alkyl groups having from 1 to 18 carbon atoms and benzyl radicals;

R₁ and R₂, which may be identical or different, are chosen from hydrogen and alkyl groups having from 1 to 6 carbon atoms;

[X]—is chosen from methosulphate anions and halides, such as chloride and bromide.

The copolymers of the family (1) also contain at least one comonomer unit which can be chosen from the family of acrylamides, methacrylamides, diacetone acrylamides, acrylamides and methacrylamides substituted on a nitrogen with substituents chosen from lower alkyls, acrylic and methacrylic acids and esters thereof, vinyllactams such as vinylpyrrolidone and vinylcaprolactam, and vinyl esters.

Thus, among these copolymers of the family (1), mention may be made of:

copolymers of acrylamide and of dimethylaminoethyl methacrylate quaternized with at least one group chosen from dimethyl sulphates and dimethyl halides, such as the one sold under the name Hercofloc by the company Hercules,

copolymers of acrylamide and of methacryloyloxyethyltrimethylammonium chloride, described, for example, in patent application EP-A-080,976, and sold under the name Bina Quat P 100 by the company Ciba Geigy, the disclosure of which is specifically herein incorporated by reference,

copolymers of acrylamide and of methacryloyloxyethyltrimethylammonium methosulphate, sold under the name Reten by the company Hercules,

quaternized and non-quaternized vinylpyrrolidone/dialkylaminoalkyl acrylate and methacrylate copolymers, such as the products sold under the name “Gafquat” by the company ISP, such as, for example, “Gafquat 734” and “Gafquat 755” and alternatively the products known as “Copolymer 845, 958 and 937”. These polymers are described in detail in French patents FR-A-2 077 143 and FR-A-2 393 573, the disclosures of all of which are specifically herein incorporated by reference,

dimethylaminoethyl methacrylate/vinylcaprolactam/vinylpyrrolidone terpolymers, such as the product sold under the name Gaffix VC 713 by the company ISP, and

quaternized vinylpyrrolidone/dimethylaminopropylmethacrylamide copolymers, such as the product sold under the name “Gafquat HS 100” by the company ISP;

(2) quaternized polysaccharides described, for example, in U.S. Pat. No. 3,589,578 and U.S. Pat. No. 4,031,307, (e.g., such as guar gums containing trialkylammonium cationic groups), the disclosure of both of the above patents are specifically herein incorporated by reference;

Such products are sold, for example, under the trade names Jaguar C13S, Jaguar C15 and Jaguar C17 by the company Meyhall.

(3) quaternary copolymers of vinylpyrrolidone and of vinylimidazole, such as the products sold by BASF under the name Luviquat TFC;

(4) chitosans and salts thereof;

the salts which can be used are, for example, chitosan acetate, lactate, glutamate, gluconate and pyrrolidonecarboxylate.

Among these compounds, mention may be made, for example, of chitosan having a degree of deacetylation of 90.5% by weight, sold under the name Kytan Brut Standard by the company Aber Technologies, and chitosan pyrrolidonecarboxylate sold under the name Kytamer PC by the company Amerchol.

(5) cationic cellulose derivatives such as, for example, copolymers of at least one of cellulose and of cellulose derivatives grafted with water-soluble monomers comprising quaternary ammonium radicals, and disclosed, for example, in U.S. Pat. No. 4,131,576, the disclosure of which is specifically herein incorporated by reference, examples of which may include hydroxyalkylcelluloses, such as, for instance, at least one group chosen from hydroxymethyl-, hydroxyethyl- and hydroxypropylcelluloses grafted with at least one group chosen from methacryloyloxyethyltrimethylammonium salts, methacrylamidopropyltrimethylammonium salts and dimethyldiallylammonium salts.

The products sold corresponding to this definition are, for example, the products sold under the name “Celquat L 200” and “Celquat H 100” by the company National Starch.

The anionic fixing polymers generally used are polymers containing groups derived from at least one acid chosen from carboxylic acid, sulphonic acid and phosphoric acid and have a molecular weight ranging approximately from 500 to 5 000 000.

1) The carboxylic groups are at least one group chosen from unsaturated monocarboxylic and dicarboxylic acid monomers such as those corresponding to the formula:

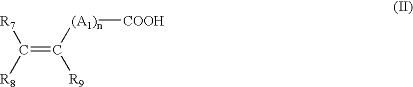

wherein n is an integer from 0 to 10, A₁ denotes a methylene group, optionally connected to the carbon atom of the unsaturated group and alternatively to a neighbouring methylene group when n is greater than 1, via a hetero atom such as oxygen and sulphur, R₇ is chosen from hydrogen, phenyl, and benzyl groups, R₈ is chosen from hydrogen, lower alkyl and carboxyl groups, R₉ is chosen from hydrogen, lower alkyl groups, —CH₂—COOH, phenyl and benzyl groups.

In the above-mentioned formula, a lower alkyl radical may be, for example, a group having 1 to 4 carbon atoms, such as methyl and ethyl.

The anionic fixing polymers containing carboxylic groups may be, for example:

A) acrylic and methacrylic acid homopolymers, acrylic and methacrylic acid copolymers, and salts of any of the above, such as, for example, the products sold under the names Versicol E and Versicol K by the company Allied Colloid and Ultrahold by the company BASF. The copolymers of acrylic acid and of acrylamide sold in the form of their sodium salt under the names Reten 421, 423 and 425 by the company Hercules, and the sodium salts of polyhydroxycarboxylic acids may be used.

B) copolymers of at least one monomer chosen from acrylic and methacrylic acids and of at least one monomer chosen from monoethylenic monomers such as ethylene, styrene, vinyl esters, acrylic and methacrylic acid esters, optionally grafted onto polyalkylene glycols such as polyethylene glycol and optionally crosslinked. Such polymers are described, for example, in French patent FR-A-1 222 944 and German patent application DE-A-2 330 956, the disclosures of which are herein incorporated by reference, the copolymers of this type containing optionally N-alkylated and hydroxyalkylated acrylamide units in their chain as described, for example, in Luxembourg patent applications 75370 and 75371, the disclosures of which are herein incorporated by reference, as well as those sold under the name Quadramer by the company American Cyanamid. Mention may also be made of copolymers of acrylic acid and of C₁-C₄ alkyl methacrylate and terpolymers of vinylpyrrolidone, of acrylic acid and of methacrylate of C₁-C₂₀ alkyl, for example of lauryl, such as the product sold by the company ISP under the name Acrylidone LM and methacrylic acid/ethyl acrylate/tert-butyl acrylate terpolymers such as the product sold under the name Luvimer 100 P by the company BASF.

C) copolymers derived from crotonic acid such as those containing at least one unit chosen from vinyl acetate and propionate units in their chain and optionally other monomers such as allylic esters and methallylic esters, vinyl ethers and vinyl esters of linear and branched saturated carboxylic acids with long hydrocarbon chains such as those containing at least 5 carbon atoms, it being possible for these polymers optionally to be grafted and crosslinked, as well as to be alternatively vinyl, allylic and methallylic esters of α- and β-cyclic carboxylic acids. Such polymers are described, inter alia, in French patents FR-A-1 222 944, FR-A-1 580 545, FR-A-2 265 782, FR-A-2 265 781, FR-A-1 564 110 and FR-A-2 439 798, the disclosures of which are herein incorporated by reference. Commercial products falling into this class are the resins 28-29-30, 26-13-14 and 28-13-10 sold by the company National Starch.

D) copolymers derived from C₄-C₈ monounsaturated carboxylic acids and anhydrides chosen from:

copolymers derived from (i) at least one group chosen from maleic, fumaric and itaconic acids and anhydrides and (ii) at least one monomer chosen from vinyl esters, vinyl ethers, vinyl halides, phenylvinyl derivatives, acrylic acid and its esters, the anhydride functions of these copolymers optionally being at least one of monoesterified and monoamidated. Such polymers are described, for example, in U.S. Pat. No. 2,047,398, U.S. Pat. No. 2,723,248 and U.S. Pat. No. 2,102,113 and GB patent GB-A-0 839 805, the disclosures of which are incorporated herein by reference, and further examples include those sold under the names Gantrez AN and Gantrez ES by the company ISP,

copolymers derived from (i) at least one of maleic, citraconic and itaconic anhydrides and (ii) at least one monomer chosen from allylic and methallylic esters optionally containing at least one unit derived from acrylamide, methacrylamide, α-olefin, acrylic ester, methacrylic ester, acrylic acid, methacrylic acid and vinylpyrrolidone in their chain,

the anhydride functions of these copolymers optionally being at least one of monoesterified and monoamidated.

These polymers are described, for example, in French patents FR-A-2 350 384 and FR-A-2 357 241, the disclosures of which are incorporated herein by reference.

E) polyacrylamides containing carboxylate groups.

The polymers comprising sulphonic groups are polymers containing at least one unit chosen from vinylsulphonic, styrenesulphonic, naphthalenesulphonic and acrylamidoalkylsulphonic units.

These polymers can be chosen, for example, from:

polyvinylsulphonic acid salts having a molecular weight ranging generally from 1 000 to 100 000, as well as the copolymers with an unsaturated comonomer such as acrylic and methacrylic acids and their esters, as well as acrylamide and its derivatives, vinyl ethers and vinylpyrrolidone;

polystyrenesulphonic acid salts, the sodium salts having a molecular weight of about 500 000 or about 100 000, which are sold respectively under the names Flexan 500 and Flexan 130 by National Starch. These compounds are described in patent FR-A-2 198 719, the disclosure of which is incorporated herein by reference;

polyacrylamidesulphonic acid salts, such as those mentioned in U.S. Pat. No. 4,128,631, the disclosure of which is incorporated herein by reference, and further such as polyacrylamidoethylpropanesulphonic acid sold under the name Cosmedia Polymer HSP 1180 by Henkel.

According to the invention, the anionic fixing polymers are, for example, chosen from acrylic acid copolymers, such as the acrylic acid/ethyl acrylate/N-tert-butylacrylamide terpolymer sold under the name Ultrahold Strong by the company BASF, copolymers derived from crotonic acid, such as vinyl acetate/vinyl tert-butylbenzoate/crotonic acid terpolymers and the crotonic acid/vinyl acetate/vinyl neododecanoate terpolymers sold under the name Resin 28-29-30 by the company National Starch, polymers derived from maleic, fumaric and itaconic acids with vinyl esters, maleic, fumaric and itaconic anhydrides with vinyl esters, vinyl ethers, vinyl halides, phenylvinyl derivatives and acrylic acid and esters thereof, such as the methyl vinyl ether/monoesterified maleic anhydride copolymer sold under the name Gantrez ES 425 by the company ISP, the copolymers of methacrylic acid and of methyl methacrylate sold under the name Eudragit L by the company Rohm Pharma, the copolymer of methacrylic acid and of ethyl acrylate sold under the names Luvimer MAEX and Luvimer MAE by the company BASF and the vinyl acetate/crotonic acid copolymer sold under the name Luviset CA 66 by the company BASF and the vinyl acetate/crotonic acid copolymer grafted with polyethylene glycol sold under the name Aristoflex A by the company BASF.

Further examples of anionic fixing polymers include those chosen from the methyl vinyl ether/monoesterified maleic anhydride copolymer sold under the name Gantrez ES 425 by the company ISP, the acrylic acid/ethyl acrylate/N-tert-butylacrylamide terpolymer sold under the name Ultrahold Strong by the company BASF, the copolymers of methacrylic acid and of methyl methacrylate sold under the name Eudragit L by the company Rohm Pharma, the vinyl acetate/vinyl tert-butylbenzoate/crotonic acid terpolymers and the crotonic acid/vinyl acetate/vinyl neododecanoate terpolymers sold under the name Resin 28-29-30 by the company National Starch, the copolymer of methacrylic acid and of ethyl acrylate sold under the names Luvimer MAEX and Luvimer MAE by the company BASF and the vinylpyrrolidone/acrylic acid/lauryl methacrylate terpolymer sold under the name Acrylidone LM by the company ISP.

The amphoteric fixing polymers which can be used in accordance with the invention can be chosen from polymers containing units B and C distributed randomly in the polymer chain, in which B denotes a unit derived from a monomer containing at least one basic nitrogen atom and C denotes a unit derived from an acid monomer containing at least one group chosen from carboxylic and sulphonic groups, and further wherein B and C, which may be identical or different, can alternatively denote groups derived from carboxybetaine and sulphobetaine zwitterionic monomers;

B and C can also denote a cationic polymer chain containing at least one of primary, secondary, tertiary and quaternary amine groups, in which at least one of the amine groups bears at least one group chosen from carboxylic and sulphonic group connected via hydrocarbon radicals and alternatively B and C form part of a chain of a polymer containing at least one α, β-dicarboxylic ethylene unit in which one of the carboxylic groups has been made to react with a polyamine containing at least one group chosen from primary and secondary amine groups.

Further examples of amphoteric fixing polymers corresponding to the definition given above may be chosen from the following polymers:

(1) polymers resulting from the copolymerization of at least one monomer chosen from vinyl compounds bearing carboxylic groups such as, for example, acrylic acid, methacrylic acid, maleic acid, α-chloroacrylic acid, and of at least one monomer chosen from basic monomers chosen from substituted vinyl compounds containing at least one basic atom, such as, for example, dialkylaminoalkyl methacrylate and acrylate, dialkylaminoalkylmethacrylamides and -acrylamides. Such compounds are described in U.S. Pat. No. 3,836,537, the disclosure of which is specifically herein incorporated by reference.

(2) polymers containing units derived from:

a) at least one monomer chosen from acrylamides and methacrylamides substituted on a nitrogen with alkyl radicals,

b) at least one acidic comonomer containing at least one reactive carboxylic group, and

c) at least one basic comonomer chosen from, for example, esters containing at least one amine substituent chosen from primary, secondary, tertiary and quaternary amine substituents of acrylic and methacrylic acids and the product of quaternization of dimethylaminoethyl methacrylate with at least one of dimethyl and diethyl sulphate.

Examples of the N-substituted acrylamides and methacrylamides include groups in which the alkyl radicals contain from 2 to 12 carbon atoms, such as, N-ethylacrylamide, N-tert-butylacrylamide, N-tert-octylacrylamide, N-octylacrylamide, N-decylacrylamide, N-dodecylacrylamide and the corresponding methacrylamides.

The acidic comonomers are chosen, for example, from acrylic acid, methacrylic acid, crotonic acid, itaconic acid, maleic acid and fumaric acid and alkyl monoesters, having 1 to 4 carbon atoms, of maleic acids, fumaric acids and anhydrides.

Examples of the basic comonomers are aminoethyl, butylaminoethyl, N,N′-dimethylaminoethyl and N-tert-butylaminoethyl methacrylates.

The copolymers whose CTFA (4th edition, 1991) name is octylacrylamide/acrylates/butylaminoethyl methacrylate copolymer, such as the products sold under the name Amphomer and Lovocryl 47 by the company National Starch, may be used.

(3) crosslinked and alkylated polyamino amides which may be at least partially derived from polyamino amides of general formula III:

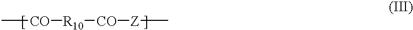

in which R₁₀ represents a divalent radical derived from at least one group chosen from dicarboxylic aromatic acids, saturated dicarboxylic acid, mono- and dicarboxylic aliphatic acids containing an ethylenic double bond, esters of a lower alkanol, having 1 to 6 carbon atoms, of these acids, and a radical derived from the addition of any one of the said acids to at least one of a bis(primary) and bis(secondary) amine, and Z denotes a bis(primary), mono- and bis(secondary) polyalkylene-polyamine radical and, for example, may represent:

a) in proportions generally ranging from 60 to 100 mol %, the radical IV

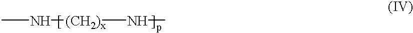

where x=2 and p is chosen from2 and 3, and alternatively x=3 and p=2

this radical being derived from at least one group chosen from diethylenetriamine, from triethylenetetraamine and from dipropylenetriamine;

b) in proportions generally ranging from 0 to 40 mol %, radicals chosen from a radical (IV) above in which x=2 and p=1 and which is derived from ethylenediamine, and a radical derived from piperazine:

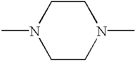

c) in proportions of from 0 to 20 mol %, the —NH(CH₂)₆—NH— radical derived from hexamethylenediamine.

These polyamino amides may be crosslinked by addition of a difunctional crosslinking agent chosen from epihalohydrines, diepoxides, dianhydrides and bis-unsaturated derivatives, using from 0.025 to 0.35 mol of crosslinking agent per amine group of the polyamino amide and alkylated by the action of at least one compound chosen from acrylic acid, chloroacetic acid and alkane sultones, and salts thereof.

The saturated carboxylic acids are, for example, chosen from acids having 6 to 10 carbon atoms, such as adipic acid, 2,2,4-trimethyladipic acid and 2,4,4-trimethyladipic acid, terephthalic acid, acids containing an ethylenic double bond such as, for example, acrylic acid, methacrylic acid and itaconic acid.

The alkane sultones used in the alkylation are, for example at least one of propane sultone and butane sultone, and the salts of the alkylating agents are, for example, at least one of sodium and potassium salts.

(4) polymers containing zwitterionic units of formula V:

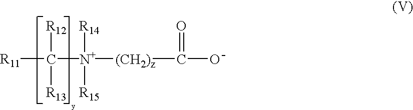

in which R₁₁ is chosen from polymerizable unsaturated groups such as acrylate, methacrylate, acrylamide and methacrylamide groups, y and z are chosen from an integer from 1 to 3, R₁₂ and R₁₃, which may be identical or different, are chosen from hydrogen, methyl, ethyl and propyl, R₁₄ and R₁₅, which may be identical or different, are chosen from hydrogen and alkyl radicals such that the sum of the carbon atoms in R₁₄ and R₁₅ does not exceed 10.

The polymers comprising such units can also contain units derived from non-zwitterionic monomers such as dimethyl acrylate, diethylaminoethyl acrylate, dimethyl methacrylate, diethylaminoethyl methacrylate, alkyl acrylates, alkyl methacrylates, acrylamides, methacrylamides and vinyl acetate.

By way of example, mention may be made of the copolymer of methyl methacrylate/methyl dimethylcarboxymethylammonioethylmethacrylate such as the product sold under the name Diaformer Z301 by the company Sandoz.

(5) polymers comprising chitosan containing units corresponding to the following formulae:

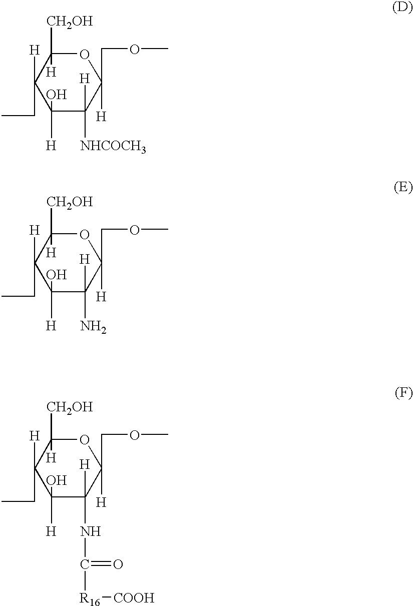

the unit D being present in proportions generally ranging from 0 to 30%, the unit E in proportions generally ranging from 5 to 50% and the unit F in proportions generally ranging from 30 to 90%, it being understood that, in this unit F, R₁₆ represents a radical of formula:

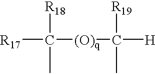

wherein, if q=0, R₁₇, R₁₈ and R₁₉, which may be identical or different, are chosen from hydrogen, methyl, hydroxyl, acetoxy and amino residues, monoalkylamine residues and dialkylamine residues which are optionally interrupted by at least one nitrogen atom and optionally substituted with at least one group chosen from amine, hydroxyl, carboxyl, alkylthio and sulphonic groups, alkylthio residues in which the alkyl group bears amino residues, at least one of the radicals R₁₇, R₁₈ and R₁₉ being, in this case, hydrogen;

and, if q=1, R₁₇, R₁₈ and R₁₉ each represent hydrogen, as well as the acid and base salts formed by any of the above compounds.

(6) polymers derived from the N-carboxyalkylation of chitosan, such as N-carboxymethylchitosan and N-carboxybutylchitosan sold under the name “Evalsan” by the company Jan Dekker.

(7) polymers corresponding to the general formula (VI) which are described, for example, in French patent FR-A-1 400 366, the disclosure of which is specifically herein incorporated by reference:

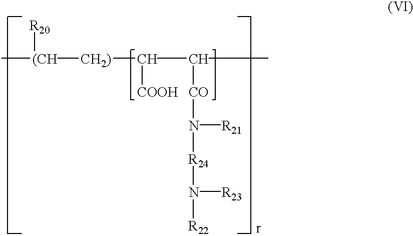

in which r is chosen such that the number-average molecular weight of said polymer ranges from 500 to 6,000,000, such as from 1000 to 1,000,000, R₂₀ is chosen from hydrogen, CH₃O, CH₃CH₂O and phenyl radicals, R₂₁ is chosen from hydrogen and lower alkyl radicals such as methyl and ethyl, R₂₂ is chosen from hydrogen and lower alkyl radicals such as methyl and ethyl, R₂₃ is chosen from lower alkyl radicals such as methyl, ethyl and radicals corresponding to the formula: —R₂₄—N(R₂₂)₂, wherein R₂₄ is chosen from —CH₂—CH₂—, —CH₂—CH₂—CH₂— and —CH₂—CH(CH₃)— radicals, and wherein R₂₂ is defined as above,

as well as the higher homologues of these radicals containing up to 6 carbon atoms.

(8) amphoteric polymers of the type —D—X—D—X— chosen from:

a) polymers obtained by the reaction of at least one of chloroacetic acid and sodium chloroacetate with compounds containing at least one unit of formula:

—D—X—D—X—D— (VII)

where D denotes a radical

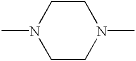

and X is chosen from E and E′, wherein E and E′, which may be identical or different, denote divalent radicals, such as divalent alkylene radicals containing at least one chain chosen from straight and branched chains with up to about 7 carbon atoms in the main chain, which may be optionally substituted with hydroxyl groups and which can contain, in addition to the oxygen, nitrogen and sulphur atoms, 1 to 3 rings, which may be identical or different, chosen from aromatic and heterocyclic rings; the oxygen, nitrogen and sulphur atoms being present in the form of groups chosen from ether, thioether, sulphoxide, sulphone, sulphonium, alkylamine, alkenylamine, hydroxyl, benzylamine, amine oxide, quaternary ammonium, amide, imide, alcohol, ester and urethane groups.

b) polymers of formula:

—D—X—D—X— (VII′)

where D denotes a radical

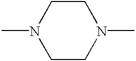

and X is chosen from E and E′, providing there is at least one E′; wherein E has the meaning given above and E′ denotes divalent radicals, such as divalent alkylene radicals chosen from straight and branched chains having up to 7 carbon atoms in the main chain, which may be optionally substituted with at least one hydroxyl radical and which contains at least one nitrogen atom, the nitrogen atom being substituted with an alkyl chain which is optionally interrupted by oxygen atoms and necessarily containing at least one carboxyl function and at least one hydroxyl function and betainized by reaction with chloroacetic acid and sodium chloroacetate.

(9) (C₁-C₅)alkyl vinyl ether/maleic anhydride copolymers partially modified by processes chosen from semiamidation with an N,N-dialkylaminoalkylamine such as N,N-dimethylaminopropylamine and semiesterification with an N,N-dialkanolamine. These copolymers can also contain other vinyl comonomers such as vinylcaprolactam.

Examples of amphoteric fixing polymers include those of family (3), such as the copolymers whose CTFA name is octylacrylamide/acrylates/butylaminoethyl methacrylate copolymer, such as the products sold under the names Amphomer, Amhomer LV 71 and Lovocryl 47 by the company National Starch and those of family (4) such as the copolymer of methyl methacrylate/methyl dimethylcarboxymethylammonioethylmethacrylate, sold, for example, under the name Diaformer Z301 by the company Sandoz.

The nonionic fixing polymers which can be used according to the present invention are chosen, for example, from:

vinylpyrrolidone homopolymers;

copolymers of vinylpyrrolidone and of vinyl acetate;

polyalkyloxazolines such as the polyethyloxazolines sold by the company Dow Chemical under the names Peox 50 000, Peox 200 000 and Peox 500 000;

vinyl acetate homopolymers, such as the product sold under the name Appretan EM by the company Hoechst, and the product sold under the name Rhodopas A 012 by the company Rhône-Poulenc;

copolymers of vinyl acetate and of acrylic ester; such as the product sold under the name Rhodopas AD 310 by Rhône-Poulenc;

copolymers of vinyl acetate and of ethylene, such as the product sold under the name Appretan TV by the company Hoechst;

copolymers of vinyl acetate and of maleic ester, for example of dibutyl maleate, such as the product sold under the name Appretan MB Extra by the company Hoechst;

copolymers of polyethylene and of maleic anhydride;

alkyl acrylate homopolymers and alkyl methacrylate homopolymers, such as the product sold under the name Micropearl RQ 750 by the company Matsumoto and the product sold under the name Luhydran A 848 S by the company BASF;

acrylic ester copolymers such as, for example, copolymers of alkyl acrylates and of alkyl methacrylates, such as the products sold by the company Rohm & Haas under the names Primal AC-261 K and Eudragit NE 30 D, by the company BASF under the names Acronal 601, Luhydran LR 8833 and Luhydran LR 8845, and by the company Hoechst under the names Appretan N 9213 and Appretan N 9212;

copolymers of acrylonitrile and of nonionic monomers chosen, for example, from butadiene and alkyl (meth)acrylates; mention may be made of the products sold under the names Nipol LX 531 B by the company Nippon Zeon and those sold under the name CJ 0601 B by the company Rohm & Haas;

polyurethanes, such as the products sold under the names Acrysol RM 1020 and Acrysol RM 2020 by the company Rohm & Haas, and the products Uraflex XP 401 UZ and Uraflex XP 402 UZ by the company DSM Resins;

copolymers of alkyl acrylate and of urethane, such as the product 8538-33 by the company National Starch;

polyamides, such as the product Estapor LO 11 sold by the company Rhône-Poulenc.

The alkyl radicals of the nonionic polymers have from 1 to 6 carbon atoms except where otherwise mentioned.

Examples of nonionic polymers suitable for preparing the compositions in accordance with the invention are those chosen from:

vinyllactam copolymers, such as copolymers of vinylpyrrolidone and of vinyl acetate, and vinylpyrrolidone/vinyl acetate/vinyl propionate copolymers

the polyvinylcaprolactam Luviskol Plus (BASF)

vinyl acetate homopolymers such as Appretan EM (Hoechst) and Rhodopas A 012 (Rhône-Poulenc)

polyalkyloxazolines such as Peox 50 000 and Peox 500 000 (Dow Chemical)

copolymers of vinyl acetate and of acrylic ester such as Rhodopas AD 310 (Rhône-Poulenc)

copolymers of vinyl acetate and of ethylene, such as Appretan TV (Hoechst)

copolymers of vinyl acetate and of maleic ester, such as Appretan MB Extra (Hoechst)

alkyl acrylate homopolymers and alkyl methacrylate homopolymers, such as Luhydran A 848 S (BASF)

acrylic ester copolymers such as Primal AC-261 K (Rohm & Haas), Acronal 601 (BASF) and Appretan N 9213 (Hoechst)

copolymers of acrylonitrile and of a nonionic monomer such as CJ 0601 B (Rohm & Haas)

polyurethanes such as Acrysol RM 1020 and Acrysol RM 2020 (Rohm & Haas)

copolymers of alkyl acrylate and of urethane, such as 8538-33 (National Starch)

polyamides such as Estapor LO 11 (Rhône-Poulenc).

According to the invention, it is also possible to use fixing polymers of grafted silicone type comprising polysiloxane portions and portions comprised of a non-silicone organic chain, one of the two portions constituting the main chain of the polymer, the other being grafted onto the said main chain. These polymers are described, for example, in patent applications EP-A-0 412 704, EP-A-0 412 707, EP-A-0 640 105 and WO 95/00578, EP-A-0 582 152 and WO 93/23009 and U.S. Pat. No. 4,693,935, U.S. Pat. No. 4,728,571 and U.S. Pat. No. 4,972,037, the disclosures of all of which are specifically herein incorporated by reference. These polymers are, for example, chosen from anionic and nonionic polymers.

Such polymers are, for example, copolymers which can be obtained by radical polymerization from the monomer mixture comprising:

a) an amount generally ranging from 50 to 90% by weight of tert-butyl acrylate;

b) an amount generally ranging from 0 to 40% by weight of acrylic acid; and

c) an amount generally ranging from 5 to 40% by weight of silicone macromer of formula:

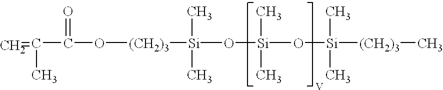

with v being a number generally ranging from 5 to 700; the weight percentages being calculated relative to the total weight of the monomers.

Other examples of grafted silicone polymers include polydimethylsiloxanes (PDMSs) onto which are grafted, via a thiopropylene-type connecting chain, mixed polymer units of the poly(meth)acrylic acid type and of the polyalkyl (meth)acrylate type and polydimethylsiloxanes (PDMSs) onto which are grafted, via a thiopropylene-type connecting chain, polymer units of the polyisobutyl (meth)acrylate type. The fixing polymers may also be chosen from functionalized and non-functionalized, silicone and non-silicone polyurethanes.

The polyurethanes suitable for the present invention are those disclosed in patents EP-A-0 751 162, EP-A-0 637 600, FR-A-2 743 297 and EP-A-0 648 485, of L'Oreal, and patents EP-A-0 656 021 and WO 94/03510 from the company BASF and EP-A-0 619 111 from the company National Starch, the disclosures of all of which are specifically herein incorporated by reference.

The insoluble non-lamellar pulverulent materials (c) may be chosen from the following products, it being understood that although several forms are possible, only the non-lamellar forms are included in the invention:

talc,

kaolin, which is a hydrated aluminium silicate,

Nylon powders, for example the product sold under the name “Orgasol 2002 ED NAT COS” by the company Atochem,

polyethylene powders, for example the product sold under the name “Coathylene HA 1681” by the company Plast Labor,

poly-β-alanine powders,

polyfluoro powders, such as polytetrafluoroethylene powders, for example the product sold under the name “MP 1400” by the company Dupont de Nemours,

acrylic copolymer powders, such as the products sold under the name “Polytrap Q5 6603” by the company Dow Chemical,

polystyrene powders, such as the products sold under the name “Polysphere 3 000 SP” by the company Presperese,

polyester powders,

expanded microspheres made of thermoplastic material, for example the product sold under the name “Expancel 551 DE” by the company Expancel,

silicone resin microbeads (for example Tospearls from the company Toshiba),

zinc oxide and titanium oxide,

aluminium oxide, zirconium oxide and cerium oxide,

precipitated calcium carbonate, magnesium carbonate and magnesium hydrocarbonate,

hydroxyapatite,

metal soaps derived from organic carboxylic acids containing from 8 to 22 carbon atoms, such as from 12 to 18 carbon atoms, for example zinc stearate, magnesium stearate, lithium stearate, zinc laurate and magnesium myristate,

silicas,

hydrophilic polymer powders which are of synthetic origin, such as polyacrylates, for example the product sold under the name “Micropearl M 100” by the company Matsumoto,

acrylic polyamides such as the products sold by the company Oris,

insoluble polyurethanes such as the product sold under the name “Plastic Powder D 800” by the company Toshnu, and

porous cellulose microspheres.

The pulverulent materials may also be materials which have been made hydrophilic by processes such as, for example, coating processes and chemical grafting processes with materials such as chitosan, titanium dioxide, silica and hydrophilic polymers, such as sulphonic polyesters and polyquaternary ammoniums.

By way of example, mention may be made of the chitosan-coated talc sold by Daito under the name “Talc CT 2 MSA”.

It is also possible to select hydrophobic pulverulent materials, chosen from pulverulent materials of hydrophobic and hydrophilic natures. In the latter case, they are made hydrophobic by processes such as coating processes and chemical grafting processes with products such as silicones, amino acids, metal soaps, fluoro derivatives, mineral oils, lecithin, isopropyl triisostearyl titanate, polyethylene, collagen, polyacrylates and derivatives thereof.

By way of example, mention may be made of the silica microbeads coated with polymethylhydrogenosiloxane which are sold under the trade name “Silica SI SB 700” by Miyoshi and the sericite coated with methicone/hydrogenated egg oil which is sold under the trade name “Sericite SNI S100” by Miyoshi.

The particle size of the pulverulent material may be, for example, in the general range of from 10 nm to 100 μm.

The “particle size” is the maximum distance which it is possible to measure between two diametrically opposite points of the particle. This size is measured, for example, using a laser granulometer.

Advantageously, the thickener (a) is present in the composition in accordance with the invention at a relative weight concentration generally ranging from 0.01% and 5%, such as from 0.1% to 2%.

The non-crosslinked fixing polymer (b) may be present in the composition in accordance with the invention at a relative weight concentration generally ranging from 0.1% to 10%, such as from 0.5% and 5%.

The pulverulent material (c) may be present in the composition in accordance with the invention at a relative weight concentration generally ranging from 0.1% to 10%, such as from 1% to 5%.

The cosmetically acceptable medium may, for example, be comprised of at least one liquid chosen from water and cosmetically acceptable solvents such as, for example, water/solvent(s) mixtures, and further wherein said solvents may be alcohols, for example C₁-C₄ alcohols.

Among these alcohols, mention may be made of ethanol and isopropanol.

The composition of the invention can also contain at least one additive chosen from anionic, cationic, nonionic and amphoteric surfactants other than those of the invention, fragrances, screening agents, preserving agents, proteins, vitamins, provitamins, polymers, plant, mineral and synthetic oils, polyols, for instance glycols and glycerol, silicones, fatty alcohols and any other additive conventionally used in cosmetic compositions.

Needless to say, a person skilled in the art will take care to select the optional compound(s) to be added to the composition according to the invention such that the advantageous properties intrinsically associated with the composition in accordance with the invention are not substantially, adversely affected by the envisaged addition.

The compositions in accordance with the invention may be applied to the skin, the nails, the lips, the hair, the eyebrows and the eyelashes.

The compositions in accordance with the invention are suitable for both dry and wet hair, as styling products.

The invention will be illustrated more fully with the aid of the non-limiting examples which follow.

All the percentages are relative percentages by weight relative to the total weight of the composition, and a.m. means active material.

The constituents used are collated in the table below.

| | |
|---|---|
| Synthalen K | Crosslinked acrylic acid homopolymer |
| | sold by 3V |
| Gafquat | Polyquaternium 11 sold by ISP |
| Klucel EF | Hydroxypropylcellulose sold by |
| | Aqualon |
| Copolymer 845 | PVP/dimethylamino methacrylate sold |
| | by ISP |
| PVP | Polyvinylpyrrolidone |
| VA | Vinyl acetate |
| Mirasil | Dimethicone copolyol sold by |
| | Rhône-Poulenc |
| Orgasol 2002 | Nylon 12 sold by Elf Atochem |
| Expancel 551 DE | Vinyl microspheres expanded with |
| | isobutane, sold by Expancel |
| Jaguar HP 105 | Gum sold by Rhône-Poulenc |
| Kytamer | Chitosan glycolate sold by Amerhold |
| Acumist B6 | Polyethylene powder sold by Allied |
| Simulsol 165 | PEG200 glyceryl stearate sold by |
| | SEPPIC |
| Brij 78 | Alkoxylated alcohol sold by Uniquema |
| Carbopol Ultrez 10 | Crosslinked acrylic acid homopolymer |
| | sold by Goodrich |
| Procetyl AWS | Alkoxylated alcohol sold by Croda |
| Belsil DMC 6038 | Dimethicone copolyol sold by Wacker |
| | Chemie |

### EXAMPLES

Various gels in accordance with the invention have been prepared.

### Example 1

Gels A to E in accordance with the invention have been prepared.

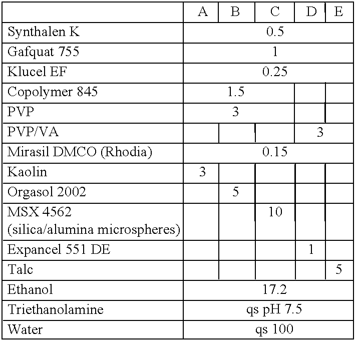

### Example 2

Gels F and G in accordance with the invention have been prepared.

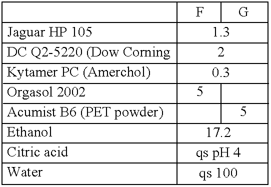

### Example 3

Gels H and I in accordance with the invention have been prepared, as follows:

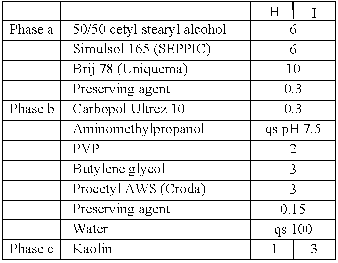

The procedure was as follows:

phase b was prepared in a manufacturing vessel. After thickening the Carbopol in water, the mixture was neutralized to pH 7.5, the other starting materials were added and the resulting mixture was heated to 80° C.;

phase a was prepared separately, by heating and dissolving the starting materials at 80° C.;

a was poured into b, followed by addition of phase c.

### Example 4

Gel J in accordance with the invention has been prepared.

| | J |
|---|---|
| Carbopol Ultrez 10 | 0.4 |
| Viscophobe DB 1000 (Amerchol) | 1.5 |
| PVP/VA | 3 |
| Belsil DMC 6038 | 0.5 |
| Kaolin | 1 |
| Ethanol | 17.2 |
| Aminomethylpropanol | qs pH 7.5 |
| Water | qs 100 |

The prepared gels did not stick to the fingers, and gave the hair a natural appearance, while at the same time fixing the hairstyle.

What is claimed is:

1. A cosmetic hair composition, comprising:
in a cosmetically acceptable medium
(a) at least one thickener,
(b) at least one fixing polymer, and
(c) at least one pulverulent material, wherein,
the at least one thickener is chosen from,
(i) natural polymers and modified natural polymers chosen from xanthan gums, scleroglucan gums, gellan gums, rhamsan gums, alginates, maltodextrins, starches and their derivatives, karaya gums, carob flours, modified and unmodified guar gums and derivatives thereof, and celluloses and derivatives thereof,
(ii) crosslinked copolymers of at least one acid chosen from acrylic acid and methacrylic acid,
(iii) crosslinked thickening polyacrylamides and
(iv) associative polymers comprising at least one hydrophilic unit and at least one fatty chain;
the at least one fixing polymer is a non-crosslinked polymer,
the at least one pulverulent material is formed from at least one non-lamellar insoluble material, and
with the proviso that said at least one thickener is not Polyquaternium-10.

2. The composition according to claim 1, wherein said at least one thickener is chosen from thickening celluloses and derivatives thereof, and guar gums and derivatives thereof.

3. The composition according to claim 1, wherein said at least one thickener is chosen from hydroxyethylcellulose, hydroxypropylcellulose and carboxymethylcellulose.

4. The composition according to claim 1, wherein said at least one thickener is chosen from xanthan gums and scleroglucan gums.

5. The composition according to claim 1, wherein said at least one thickener comprises hydroxypropylguar.

6. The composition according to claim 1, wherein said at least one thickener is chosen from acrylic acid/ethyl acrylate copolymers and carboxyvinyl polymers.

7. The composition according to claim 1, wherein said at least one thickener is chosen from:
crosslinked 2-acrylamido-2-methylpropanesulphonic homopolymers,
copolymers of acrylamide and of ammonium acrylate,
crosslinked copolymers of methacryloyloxyethyltrimethylammonium chloride and at least one monomer chosen from acrylamides and methacrylamides, and
crosslinked, at least partially neutralized copolymers of acrylamides and of 2-acrylamido-2-methylpropanesulphonic acids.

8. The composition of claim 7, wherein said copolymers of acrylamide and of ammonium acrylate are crosslinked.

9. The composition according to claim 8, wherein said at least one thickener is chosen from crosslinked copolymers of acrylamide/ammonium acrylate in a ratio of 5 percent acrylamide to 95 percent ammonium acrylate by weight crosslinked with at least one crosslinking agent containing polyolefinic unsaturation.

10. The composition according to claim 8, wherein said at least one thickener is chosen from crosslinked copolymers of acrylamide/ammonium acrylate in the form of a water-in-oil emulsion.

11. The composition according to claim 7, wherein said at least one thickener is chosen from crosslinked copolymers of acrylamide and of 2-acrylamido-2-methylpropanesulphonic acid crosslinked with compounds containing polyolefinic unsaturation.

12. The composition according to claim 7, wherein said at least one thickener is chosen from crosslinked copolymers of acrylamide and of 2-acrylamido-2-methylpropanesulphonic acid in the form of an oil-in-water emulsion.

13. The composition according to claim 7, wherein said at least one thickener is chosen from methacryloyloxyethyltrimethylammonium chloride/acrylamide copolymers which are crosslinked and in the form of a dispersion containing mineral oil.

14. The composition according to claim 7, wherein said at least one thickener is chosen from methacryloyloxyethyltrimethylammonium chloride/methacrylamide copolymers which are non-crosslinked.

15. The composition according to claim 1 wherein the associative polymers are associative polyurethanes.

16. The composition according to claim 1, wherein the at least one fixing polymer is chosen from anionic, cationic, amphoteric and nonionic fixing polymers.

17. The composition according to claim 16, wherein the at least one fixing polymer is chosen from anionic polymers chosen from:
polymers comprising carboxylic units derived from monomers chosen from unsaturated monocarboxylic and dicarboxylic acid monomers of formula: 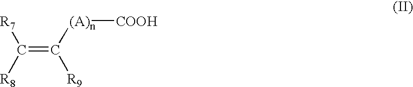
wherein n is an integer from 0 to 10, A denotes a methylene group, R₇ is chosen from hydrogen, phenyl, and benzyl groups, R₈ is chosen from hydrogen, lower alkyl and carboxyl groups, and R₉ is chosen from hydrogen, lower alkyl groups, —CH₂—COOH, phenyl and benzyl groups; and
polymers comprising units derived from sulphonic acid.

18. The composition according to claim 17, wherein said units derived from sulphonic acid are chosen from vinylsulphonic, styrenesulphonic and acrylamidoalkylsulphonic units.

19. The composition according to claim 17, wherein A is connected to the carbon atom of the unsaturated group via at least one hetero atom.

20. The composition according to claim 17, wherein A is connected to at least one neighbouring methylene group via at least one hetero atom when n is greater than 1.

21. The composition according to claim 1, wherein the at least one fixing polymer is an amphoteric polymer chosen from polymers resulting from the copolymerization of at least one monomer chosen from vinyl compounds bearing carboxylic groups and at least one basic monomer chosen from substituted vinyl compounds containing at least one basic atom.

22. The composition according to claim 1, wherein said at least one fixing polymer is chosen from amphoteric polymers comprising units derived from:
a) at least one monomer chosen from acrylamides and methacrylamides substituted on the nitrogen with an alkyl radical,
b) at least one acidic comonomer containing at least one reactive carboxylic group, and
c) at least one basic comonomer.

23. The composition according to claim 22, where said basic comonomer is chosen from esters of acrylic and methacrylic acids containing at least one amine substituent chosen from primary, secondary, tertiary and quaternary amine substituents and quaternization products of dimethylaminoethyl methacrylate with at least one of dimethyl and diethyl sulphate.

24. The composition according to claim 1, wherein said at least one fixing polymer is chosen from nonionic polymers chosen from:
vinylpyrrolidone homopolymers;
copolymers of vinylpyrrolidone and of vinyl acetate;
polyalkyloxazolines;
vinyl acetate homopolymers;
copolymers of vinyl acetate and of acrylic ester;
copolymers of vinyl acetate and of ethylene;
copolymers of vinyl acetate and of maleic ester;
copolymers of polyethylene and of maleic anhydride;
alkyl acrylate homopolymers and alkyl methacrylate homopolymers;
acrylic ester copolymers;
copolymers of acrylonitrile and of a nonionic monomer;
copolymers of alkyl acrylate and of urethane;
polyamides; and
polyurethanes.

25. The composition according to claim 24, wherein said non-ionic polymers are chosen from acrylic ester copolymers formed from at least one of alkyl acrylates and alkyl methacrylates.

26. The composition according to claim 1, wherein the at least one fixing polymer is chosen from cationic polymers chosen from homopolymers and copolymers derived from at least one group chosen from acrylic esters, methacrylic esters and amides and containing at least one of the units of the following formulae: 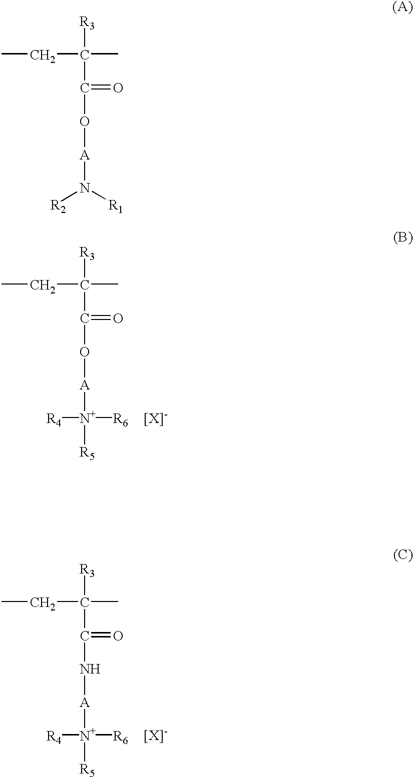
wherein:
R₃ is chosen from hydrogen and CH₃ radicals;
A is chosen from linear and branched alkyl group of 1 to 6 carbon atoms and hydroxyalkyl groups of 1 to 4 carbon atoms;
R₄, R₅ and R₆, which may be identical or different, are chosen from alkyl groups having from 1 to 18 carbon atoms and benzyl radicals;
R₁ and R₂, which may be identical or different, are chosen from hydrogen and alkyl groups having from 1 to 6 carbon atoms;
[X]—is chosen from methosulphate anions and halides.

27. The composition according to claim 1 wherein said at least one fixing polymer is chosen from cationic polymers chosen from:
copolymers of acrylamide and of dimethylaminoethyl methacrylate quaternized with dimethyl sulphate,
copolymers of acrylamide and of methacryloyloxyethyltrimethylammonium chloride,
copolymers of acrylamide and of methacryloyloxyethyltrimethylammonium methosulphate,
quaternized and non-quaternized vinylpyrrolidone/dialkylaminoalkyl acrylate and vinylpyrrolidone/dialkylaminoalkyl methacrylate copolymers,
dimethylaminoethyl methacrylate/vinylcaprolactam/vinylpyrrolidone terpolymers,
quaternized vinylpyrrolidone/dimethylaminopropylmethacrylamide copolymers,
quaternized polysaccharides,
quaternary copolymers of vinylpyrrolidone and of vinylimidazole,
chitosans and salts thereof,
cationic cellulose derivatives.

28. The composition according to claim 27, wherein said quaternized polysaccharides are chosen from guar gums containing trialkylammonium cationic groups.

29. The composition according to claim 1 wherein said at least one fixing polymer is chosen from polyurethanes.

30. The composition according to claim 29, wherein said polyurethanes are chosen from silicone polyurethanes.

31. The composition according to claim 1, wherein said at least one fixing polymer is chosen from polymers of grafted silicone comprising a polysiloxane portion and a portion comprising a non-silicone organic chain, one of the two portions constituting the main chain of the polymer, the other being grafted onto the said main chain.

32. The composition according to claim 1, wherein said at least one pulverulent material is chosen from the following non-lamellar materials:
talc,
kaolin,
Nylon powders,
polyethylene powders,
poly-β-alanine powders,
polyfluoro powders,
acrylic copolymer powders,
polystyrene powders,
polyester powders,
expanded microspheres made of thermoplastic material,
silicone resin microbeads,
zinc oxide and titanium oxide
aluminium oxide,
zirconium oxide,
cerium oxide,
precipitated calcium carbonate, magnesium carbonate and magnesium hydrocarbonate,
hydroxyapatite,
metal soaps derived from organic carboxylic acids containing from 8 to 22 carbon atoms,
silicas,
hydrophilic polymer powders which are of synthetic origin,
acrylic polyamides,
insoluble polyurethanes, and
porous cellulose microspheres.

33. The composition according to claim 32, wherein said metal soaps are derived from organic carboxylic acids containing from 12 to 18 carbon atoms.

34. The composition according to claim 32, wherein said hydrophilic polymer powders of synthetic origin are chosen from polyacrylates.

35. The composition according to claim 1, wherein said at least one thickener is present in the composition at a relative weight concentration ranging from 0.01% to 5%.

36. The composition according to claim 35, wherein said at least one thickener is present in the composition at a relative weight concentration ranging from 0.1% to 2%.

37. The composition according to claim 1, wherein said at least one fixing polymer is present in the composition at a relative weight concentration ranging from 0.1% to 10%.

38. The composition according to claim 37, wherein said at least one fixing polymer is present in the composition at a relative weight concentration ranging from 0.5% to 5%.

39. The composition according to claim 1, wherein said at least one pulverulent material is present in the composition in a relative weight concentration ranging from 0.1% to 10%.

40. The composition according to claim 39, wherein the pulverulent material is present in the composition in a relative weight concentration ranging from 1% to 5%.

41. The composition according to claim 1, wherein the cosmetically acceptable medium comprises at least one liquid chosen from water and other cosmetically acceptable solvents.

42. The composition according to claim 41, wherein the cosmetically acceptable solvents are chosen from alcohols.

43. The composition according to claim 41, wherein the cosmetically acceptable solvents are chosen from C₁-C₄ alcohols.

44. The composition according to claim 1, wherein said composition further comprises at least one additive chosen from anionic, cationic, nonionic and amphoteric surfactants, fragrances, screening agents, preserving agents, proteins, vitamins, provitamins, polymers, plant, mineral and synthetic oils, polyols, silicones, and fatty alcohols.

45. The composition according to claim 44, wherein said polyols are chosen from glycols and glycerol.

46. A gel comprising, in a cosmetically acceptable medium
(a) at least one thickener,
(b) at least one fixing polymer, and
(c) at least one pulverulent material, wherein,
the at least one thickener is chosen from,
(i) natural polymers and modified natural polymers chosen from xanthan gums, scleroglucan gums, gellan gums, rhamsan gums, alginates, maltodextrins, starches and their derivatives, karaya gums, carob flours, modified and unmodified guar gums and derivatives thereof, and celluloses and derivatives thereof,
(ii) crosslinked copolymers of at least one acid chosen from acrylic acid and methacrylic acid,
(iii) crosslinked thickening polyacrylamides and
(iv) associative polymers comprising at least one hydrophilic unit and at least one fatty chain;
the at least one fixing polymer is a non-crosslinked polymer, and
the at least one pulverulent material is formed from at least one non-lamellar insoluble material.

47. A process comprising applying a composition to hair, wherein said composition comprises, in a cosmetically acceptable medium
(a) at least one thickener,
(b) at least one fixing polymer, and
(c) at least one pulverulent material, wherein,
the at least one thickener is chosen from,
(i) natural polymers and modified natural polymers chosen from xanthan gums, scleroglucan gums, gellan gums, rhamsan gums, alginates, maltodextrins, starches and their derivatives, karaya gums, carob flours, modified and unmodified guar gums and derivatives thereof, and celluloses and derivatives thereof,
(ii) crosslinked copolymers of at least one acid chosen from acrylic acid and methacrylic acid,
(iii) crosslinked thickening polyacrylamides and
(iv) associative polymers comprising at least one hydrophilic unit and at least one fatty chain;
the at least one fixing polymer is a non-crosslinked polymer,
the at least one pulverulent material is formed from at least one non-lamellar insoluble material, and
with the proviso that said at least one thickener is not Polyquaternium-10.

48. A process for at least one of fixing and holding a hair style comprising applying an effective amount of a composition to said hair, said composition comprising:
in a cosmetically acceptable medium
(a) at least one thickener,
(b) at least one fixing polymer, and
(c) at least one pulverulent material, wherein,
the at least one thickener is chosen from,
(i) natural polymers and modified natural polymers chosen from xanthan gums, scleroglucan gums, gellan gums, rhamsan gums, alginates, maltodextrins, starches and their derivatives, karaya gums, carob flours, modified and unmodified guar gums and derivatives thereof, and celluloses and derivatives thereof,
(ii) crosslinked copolymers of at least one acid chosen from acrylic acid and methacrylic acid,
(iii) crosslinked thickening polyacrylamides and
(iv) associative polymers comprising at least one hydrophilic unit and at least one fatty chain;
the at least one fixing polymer is a non-crosslinked polymer, and
the at least one pulverulent material is formed from at least one non-lamellar insoluble material.

49. A cosmetic hair composition, comprising:
in a cosmetically acceptable medium
(a) at least one thickener,
(b) at least one fixing polymer, and
(c) at least one pulverulent material, wherein,
the at least one thickener is chosen from,
(i) natural polymers and modified natural polymers of natural microbial origin
(ii) crosslinked copolymers of at least one acid chosen from acrylic acid and methacrylic acid,
(iii) crosslinked thickening polyacrylamides and
(iv) associative polymers comprising at least one hydrophilic unit and at least one fatty chain;
the at least one fixing polymer is a non-crosslinked polymer, and
the at least one pulverulent material is formed from at least one non-lamellar insoluble material.

50. A cosmetic hair composition, comprising:
in a cosmetically acceptable medium
(a) at least one thickener,
(b) at least one fixing polymer, and
(c) at least one pulverulent material, wherein,
the at least one thickener is chosen from,
(i) natural polymers and modified natural polymers chosen from xanthan gums, scleroglucan gums, gellan gums, rhamsan gums, alginates, maltodextrins, starches and their derivatives, karaya gums, carob flours, modified and unmodified guar gums and derivatives thereof,
(ii) crosslinked copolymers of at least one acid chosen from acrylic acid and methacrylic acid,
(iii) crosslinked thickening polyacrylamides and
(iv) associative polymers comprising at least one hydrophilic unit and at least one fatty chain;
the at least one fixing polymer is a non-crosslinked polymer, and
the at least one pulverulent material is formed from at least one non-lamellar insoluble material.

51. A process comprising applying a composition to hair, wherein said composition comprises, in a cosmetically acceptable medium
(a) at least one thickener,
(b) at least one fixing polymer, and
(c) at least one pulverulent material, wherein,
the at least one thickener is chosen from,
(i) natural polymers and modified natural polymers chosen from xanthan gums, scleroglucan gums, gellan gums, rhamsan gums, alginates, maltodextrins, starches and their derivatives, karaya gums, carob flours, modified and unmodified guar gums and derivatives thereof,
(ii) crosslinked copolymers of at least one acid chosen from acrylic acid and methacrylic acid,
(iii) crosslinked thickening polyacrylamides and
(iv) associative polymers comprising at least one hydrophilic unit and at least one fatty chain;
the at least one fixing polymer is a non-crosslinked polymer, and
the at least one pulverulent material is formed from at least one non-lamellar insoluble material.
